(19) United States
(12) Patent Application Publication
(10) Pub. No.: US 2019/0159930 A1
(43) Pub. Date: May 30, 2019

(54) AN INJECTION AND EXTRACTION OPHTHALMIC DEVICE

(71) Applicant: SINGAPORE HEALTH SERVICES PTE LTD, Singapore (SG)

(72) Inventors: Marcus Han Nian ANG, Singapore (SG); Kelvin Yi Chong TEO, Singapore (SG); Anna Cheng Sim TAN, Singapore (SG)

(73) Assignee: SINGAPORE HEALTH SERVCIES PTE LTD, Singapore (SG)

(21) Appl. No.: 16/091,177

(22) PCT Filed: Apr. 21, 2017
(86) PCT No.: PCT/SG2017/050225

§ 371 Date: Oct. 4, 2018

(30) Foreign Application Priority Data
Apr. 21, 2016 (SG) 10201603169X

(51) Int. Cl. A61F 9/00; A61B 10/00; A61M 5/31; A61M 5/32

(52) U.S. Cl. CPC A61F 9/0017; A61M 5/3202; A61M 5/31; A61B 10/0045

(57) ABSTRACT

An extraction and injection ophthalmic device comprising; a housing having;
an injection port arranged to receive an injection source; a vacuum port arranged to receive an vacuum source; an eye engagement portion, and; at least one needle for selective fluid communication with one or both of the vacuum port or injection port; wherein on activation of the vacuum source the device is arranged to extract a sample through the needle, and on activation of the injection source the device is arranged to inject an agent through the needle.

### FIELD OF THE INVENTION

The present invention relates to retrieval of a bodily fluid for diagnosis thereafter injection of a therapeutic drug used for ophthalmology and the eye, and for similar purposes in other parts of the human body.

### BACKGROUND

Age-related macular degeneration (AMD) and diabetic eye disease are the leading causes of blindness in elderly patients in the world, including Singapore and other Asian countries. Anti-vascular endothelial growth factor (anti-VEGF) agents, injected directly into the eye (vitreous humor), have emerged as the standard of care for these diseases. This has been shown to be superior to the other treatment modalities: photodynamic therapy for AMD and laser therapy for DR. However, a standardised method of delivery, the frequency of the injections necessary for optimum results without over-treatment, and the ocular and systemic safety of these repeated injections over long periods of time are unclear.

While intra-vitreal injections of anti-VEGF agents is now the treatment of choice for AMD and diabetic eye disease results of clinical trials suggest that a significant proportion of patients may be sub-optimal responders to this treatment. Even those that do respond, to prevent progression or recurrence of the disease, patients may be subject to long term injections. However, multiple injections expose the patient to an increased risk of blinding infection, damage to ocular structures (e.g., retinal detachment, cataract), a higher risk of glaucoma and even systemic complications (e.g. stroke and ischaemic heart disease.

In some treatments of the eye, sampling of ophthalmic fluid (vitreous or aqueous humor) is extracted from the eye for analysis. This procedure is not commonly performed and not performed routinely in the treatment of AMD and diabetic eye disease due to the risk involved in the procedure. Currently, a sample of ophthalmic fluid is considered necessary only in severe conditions such as enophthalmitis (severe infection in the eye) or severe inflammation to ascertain a microbial diagnosis for targeted antimicrobial treatment. Thereafter, a drug is administered to the eye according to the condition. The extraction of ophthalmic fluid is conducted in a procedure separate from the delivery of the drug. At least two punctures to the posterior chamber is required in order to perform the function of extraction and delivery. Another disadvantage of the current method of intra-vitreal injection of medication into the eye is that when a fixed volume of drug is injected into an already pressurised eye, there is an increased risk of a temporary spike in intra-ocular pressure (IOP) and reflux of drug from the injection site. Therefore, sample extraction before intra-vitreal injection may potentially depressurise the eye to potentially improve safety by reducing spikes in IOP and reducing drug reflux delivering more active drug into the intra-ocular space. Previous studies have shown that the risk of glaucoma surgery is higher in eyes receiving multiple intra-vitreal injections. In addition, pre-existing glaucoma or ocular hypertension has also been shown to be an important factor for long term IOP increase. Hence, the reduction of IOP spikes in patients with concurrent glaucoma undergoing repeated intra-vitreal injections as well may be useful to prevent disease progression.

Extraction of vitreous thought a needle is challenging as the vitreous may be in a gel-like state making it impossible for suction. In addition, the gel-like state of the vitreous and adhesions to the retina may pose additional risk from traction when the device is retracted from the eye. Hence, a cutting stroke to reduce gel-like form for safer retrieval of the device is required and may provide a better yield of the sample.

Currently there is no standardised method of intra-vitreal injections. While some compounds come pre-packaged in a ready-to-use syringe, other compounds require the transfer of the compound from the vial to a syringe. In addition, for all compounds, a 30 G needle is required to be manually connected to the syringe before injection. This non-standardised technique and excessive manipulation exposes the patient to an unnecessary risk of infection. In users, who are not used to the injection technique, there may be also a higher risk of damage to ocular structures, cataract and other complications. The increased injection load is both a treatment burden for the patients and a challenge to any busy retinal practice. Safer and more efficient methods of drug administration and even training other non-medical staff to perform this procedure may be required to cope with the increasing demand. Hence, a safe, user-friendly and reliable way to extract vitreous and inject anti-VEGF agents may be of demand.

### SUMMARY OF INVENTION

In a first aspect, the invention provides an extraction and injection ophthalmic device comprising; a housing having; an injection port arranged to receive an injection source; a vacuum port arranged to receive an vacuum source; an eye engagement portion, and; at least one needle for selective fluid communication with one or both of the vacuum port or injection port; wherein on activation of the vacuum source the device is arranged to extract a sample through the needle, and on activation of the injection source the device is arranged to inject an agent through the needle.

Therefore, the invention is directed to a device having both a diagnostic and therapeutic effect capable of performing both ocular fluid biopsy (vitreous sampling) and injection of therapeutic drugs. To this end, a device according to one embodiment of the present invention may provide for a fully self-contained extraction and injection device.

The means by which the therapeutic is injected may vary. In one embodiment, a syringe may be used. Alternatively, a range of positive pressure devices may also be useful including a pump.

For the sample extraction, similarly a negative pressure device is required which may be a syringe. Alternative devices could also be used, such as sample tubes (such as a vacutainer) having an internal negative pressure arranged to draw in the sample, and a pump applying a negative pressure.

The invention may include a device having a vitreous extractor and an injector mechanism capable of extracting the vitreous fluid and injecting a drug. The device may have a single needle puncture adapted to extract the vitreous fluid and deliver a fluid source.

The injector device may have the potential to standardise the injection technique in a more safe and reproducible way. The volume of drug delivery may also be more reproducible if reflux is reduced. The extraction of vitreous fluid from the eye for rapid biomarker analysis, may allow a tailored, individualised treatment based on biomarker levels and give additional outcome measures for treatment.

The “diagnostic plus therapeutic” device-vitreous extractor-plus-injector may simultaneously perform ocular fluid biopsy (vitreous sampling) and injection of therapeutic drugs safely and easily. Biomarker analysis may be performed on the vitreous fluid extracted to determine specific dosage and injection frequency, enabling individualised and patient-tailored therapy.

The invention may provide a safe and reliable way to extract vitreous and inject anti-VEGF agents into the eye. Injections are currently performed using improvised syringes meant for venepuncture and blood sampling. It is unstable and exposes the patient to risk of infection, damage to ocular structures, cataract and even blindness, requiring a skilled doctor to perform. As patients will require regular injections over their lifetime, there may be a need for nurses or technicians to perform this routine procedure to reduce burden on hospitals and doctors.

The removal of a small amount of vitreous humor before the injection of the therapeutic may prevent a spike in intraocular pressure and so reduce complications in the eye. To this end, the device may include a shearing device for cutting through extracted vitreous humor, so as to separate the sample from the remaining material. The shearing device may be a dedicated cutting tool, or may be included as part of the sample extracting mechanism.

In one embodiment, a concentric-type shearing device may be used. Alternatively, a horizontal sliding-type shearing may be used. In this instance, in an embodiment whereby the device includes a slidable sample collection module, this sliding action may be used to shear through the excess vitreous humor.

Three main purposes of the shearing device:
To shear the gel-like viscous vitreous humor after biopsy
To prevent vitreous humor from re-entering the eye after biopsy
To prevent retinal detachment when the needle is removed from the eye after biopsy due to the viscous nature of the vitreous humor.

Whilst the description refers to use with the eye, it will be appreciated that the invention may also include a device performing the same function for other portions of the body, where extraction of a sample and injection of a therapeutic material may benefit from a self-contained device.

### BRIEF DESCRIPTION OF THE DRAWINGS

It will be convenient to further describe the present invention with respect to the accompanying drawings which illustrate possible arrangements of the invention. Other arrangements of the invention are possible, and consequently the particularity of the accompanying drawings is not to be understood as superseding the generality of the preceding description of the invention.

FIG. 1 is an isometric view of an extraction and injection ophthalmic device in accordance with an embodiment of the present invention.

FIG. 2A is a cross-sectional view of the extraction and injection ophthalmic device before actuation in accordance with an embodiment of the present invention.

FIG. 2B is a cross-sectional view of the extraction and injection ophthalmic device whereby a piston spring loaded is released in accordance with an embodiment of the present invention.

FIG. 2C is a cross-sectional view of the extraction and injection ophthalmic device whereby a sample of vitreous fluid travels along a path to a collection chamber in accordance with an embodiment of the present invention.

FIGS. 3A and 3B are views of the extraction and injection ophthalmic device where the sample of vitreous fluid finally resides in accordance with an embodiment of the present invention.

FIG. 4 is a view of the extraction and injection ophthalmic device where the sample of vitreous fluid in the collection chamber dislodged in accordance with an embodiment of the present invention.

FIG. 5A is a cross-sectional view of the extraction and injection ophthalmic device whereby a cutting stroke actuated in accordance with an embodiment of the present invention.

FIG. 5B is a cross-sectional view of the extraction and injection ophthalmic device where a plunger actuated in accordance with an embodiment of the present invention.

FIG. 6 is a detailed view of the extraction and injection ophthalmic device as shown in FIG. 5B.

FIGS. 7A to 7C are various views of an extraction and injection ophthalmic device according to a further embodiment of the present invention.

FIGS. 8A and 8B are various views of an extraction and injection ophthalmic device according to a further embodiment of the present invention.

FIGS. 9A and 9B are sequential views of the operation of the extraction and injection ophthalmic device of FIGS. 8A and 8B.

FIGS. 10A to 10C are various views of an extraction and injection ophthalmic device according to a still further embodiment of the present invention.

FIGS. 11A to 11C are sequential views of the operation of the extraction and injection ophthalmic device as shown in FIGS. 10A to 10C.

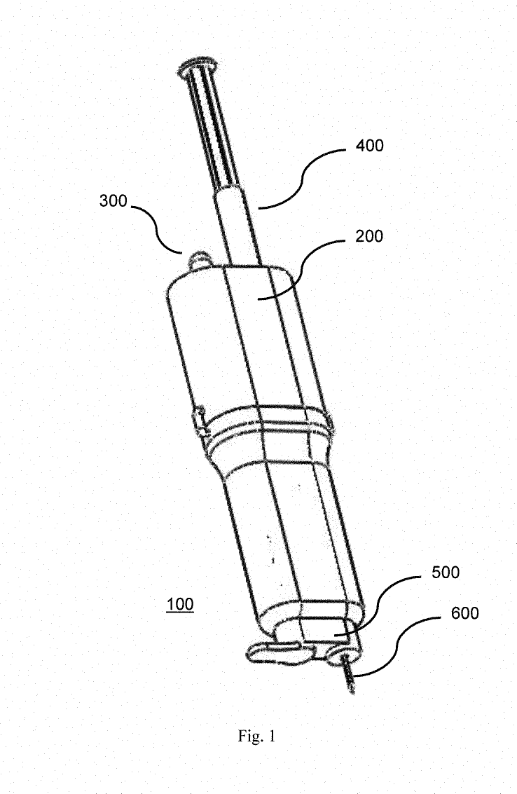

### DETAILED DESCRIPTION

Referring to the drawings, in order to provide swift extraction of a vitreous sample, cutting of gel-like state of the vitreous sample and delivery of a therapeutic drug, in one embodiment, the present invention provides a disposable handheld extraction and delivery device 100 to facilitate extraction of the vitreous sample from an eye of a patient, the device comprising a casing 200 extending from a proximal end to a distal end and at least one opening, at the distal end, a needle portion 600 having a through hole, protruding out from the at least one opening, the needle portion 600 includes at least one side hole along a predetermined length of the needle portion 600. The at least one side hole 610 of the needle portion 600 may be sized and shaped preferably to increase suction area. The at least one side hole 610 shaped in any manner preferably rectangular to maximise streamlined flow exchange. In the alternative, the at least one side hole 610 may be shaped circularly or squared.

The casing 200 comprising at least one slot portion adapted to receive an injection syringe 400 and/or a vacuum syringe 300. The device 100 may be used with either function and not limited to both the injection syringe 400 and the vacuum syringe 300. More intimately, the user may choose to only deploy the injection syringe 400 into the casing while the vacuum syringe 300 is separate. In the alternative, the vacuum syringe 300 may be deployed into the casing 200 for extraction of the vitreous sample. Both the injection syringe 400 and vacuum syringe 300 detachable from each of the at least one slot of the casing 200.

The needle portion 600 connected to an injection syringe 400 along a first slot of the at least one slot portion. The casing 200 houses a first portion of the needle portion 600 while a second portion houses the activation unit of the injection syringe 400. A portion of the activation unit protrudes externally from the proximal end.

The device 100 avoids the need to pull out the needle portion 600 from the eye by providing a pathway where a suction force from the vacuum syringe 300 and a push force from the injection syringe 400 employed in sequence. The suction force is in the range of 50 to 400 mbar. Ideally, the suction force ranges from 100 to 200 mbar. This device allows a controlled approach for retrieval of the vitreous sample and delivery of the therapeutic drug. In addition, the casing 200 includes a guide portion 700 positioned at the distal end. With the guide portion 700, the needle portion 600 does not “overrun” beyond a desired distance of the eye. The guide portion 700 protects the eye by allowing the user with a pressure indication.

In the particular embodiment, the device includes a sample collection module 500 positioned within the casing 200 close to the distal end. In the alternative, the sample collection module 500 can be detachable from the casing 200 thereby allowing the user to retrieve the vitreous sample in quick succession for analysis. Further sample processing of the vitreous sample for analysis may be required for therapeutic administration. In order to view whether the vitreous sample is collected, the sample collection module 500 is made of translucent or transparent material for visual observation of the vitreous sample being collected.

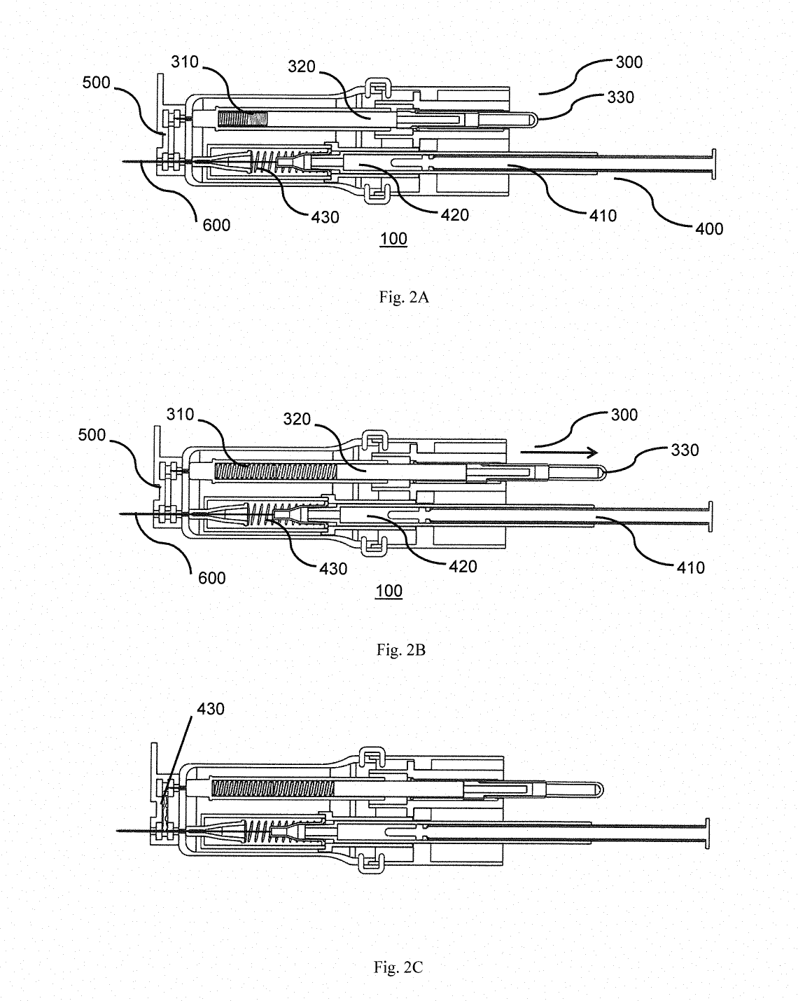

Referring to FIGS. 2A-2C describes the device 100 more particularly the vacuum syringe 300. The vacuum syringe 300 includes a vacuum spring 310, a vacuum chamber 320 and a vacuum actuator 330. In operation, the vacuum syringe 300 in a ready state or a first position includes the vacuum spring 310 in a compressed state. The ready state defines as no suction force enabled and the vacuum spring 310 in the compressed state where the vacuum chamber 320 and the vacuum actuator 330 compresses and acts onto the vacuum spring 310. The vacuum actuator 330 upon actuation releases the vacuum spring 310 from the compressed state to an uncompressed state whereby the vacuum chamber 320 is now repositioned further away from the distal end.

In use, when the vacuum spring 310 is uncompressed, a suction force is enabled to facilitate retrieval and catchment of the vitreous sample through a sample retrieval pathway passing by the sample collection module 500 and entering through at least one side hole 610 of the needle portion 600. A predetermined amount of the vitreous sample resides within the sample collection module 500 accordingly. A person skilled in the art will readily understand that it is ideal for the user to extract the vitreous sample first prior to delivery of a therapeutic drug. The collection and extraction of the vitreous sample first is to prevent accidental suction and extraction of the therapeutic drug. For illustrative purpose, the device 100 can be applied beyond the use of an eye. In addition, the device 100 can be applied any part of the body for extraction of bodily fluid collection and drug injection.

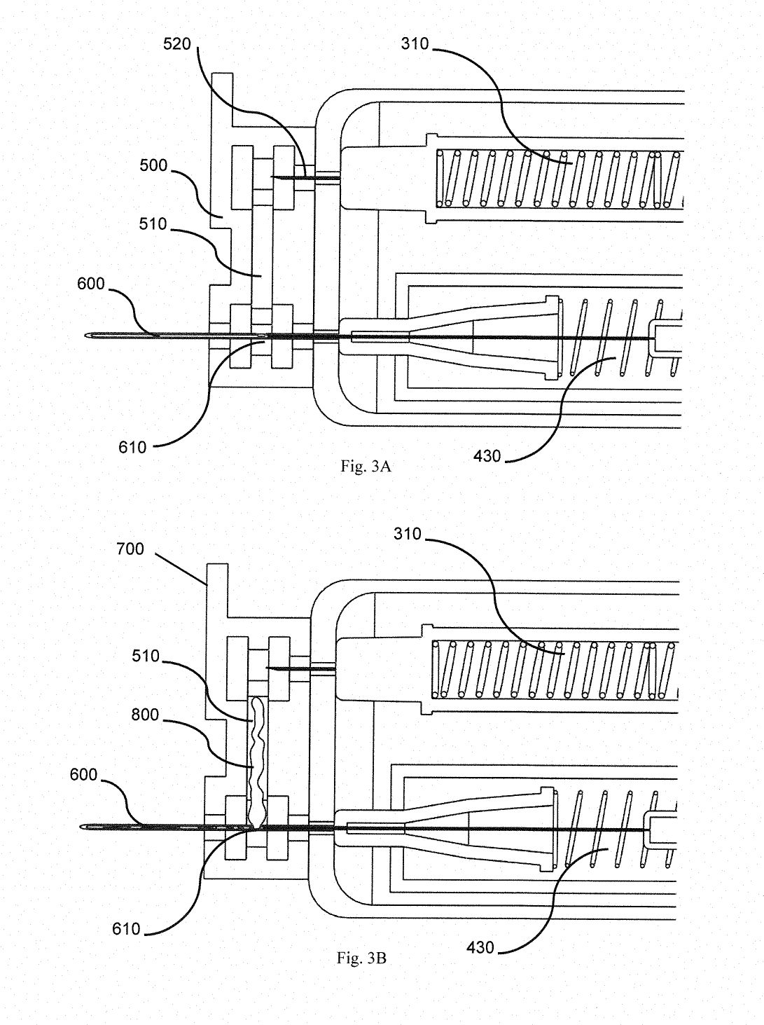

Referring now to FIGS. 3A and 3B, the sample collection module 500 describes the sample retrieval pathway where the suction force passes. More intimately, when the needle portion 600 is inserted into a body, upon depressing of the vacuum actuator 330, the vacuum spring 310 expands, the suction force first pass through a connecting tube 520 and travels to and through the at least one side hole 610. The vitreous sample 800 will reside within the sample collection module 500.

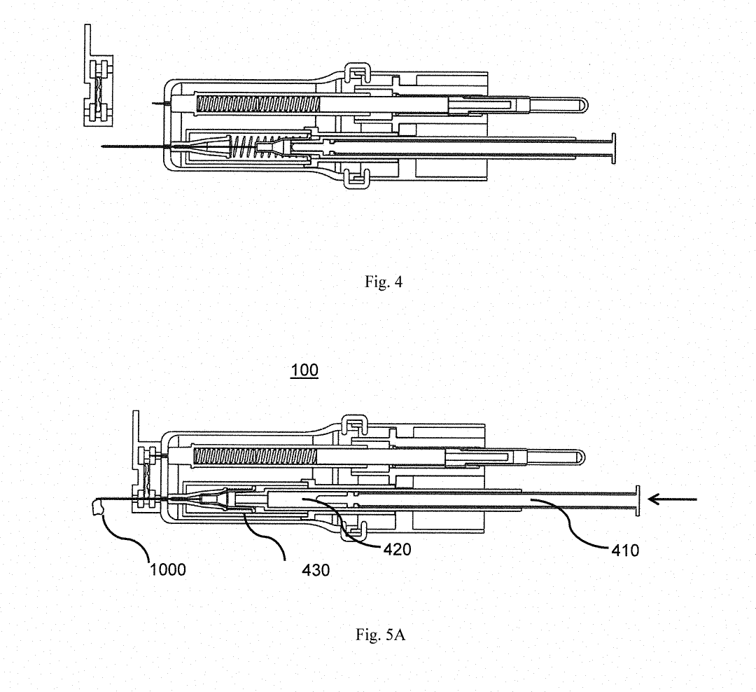

For collection of the vitreous sample 800 residing within the sample collection module 500, the user may simply push off to dislodge the sample collection module 500 away from the device 100. FIG. 4 illustrates the sample collection module 500 where the device 100 may be reusable. The sample collection module 500 may be connected to the device via a snap-fit module where the shape and size of a portion of the device fits snugly the sample collection module 500.

FIGS. 5A and 5B illustrates the delivery process of the therapeutic drug. The injection syringe 400 includes an injection plunger or actuator 410 where a portion of the injection plunger 410 disposed exteriorly from the casing 200. In use, a user pushes or advances the injection plunger 410 further forward into an elongated portion of the injection syringe 400 thereby activating an inner needle 620 disposed within the needle portion 600. The inner needle 620 profiled with a substantially curved portion to provide a cutting effect upon activation. In operation, some gel-like substance 1000 may be stuck at an opening of the needle portion 600. By breaking the gel-like substance 1000, the delivery of the therapeutic drug will be seamless since no addition blockage ensures fluid to flow smoothly.

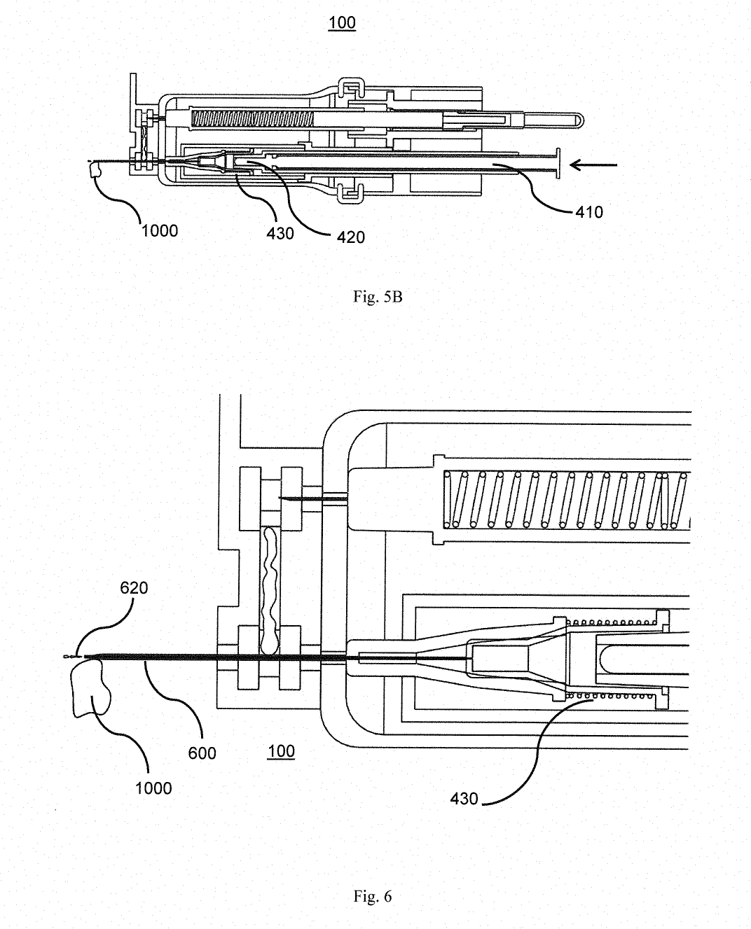

Referencing further to FIG. 6, the device 100 when in use describes the inner needle 620 being actuated away. In an original position, the inner needle 620 resides within the needle portion 600, upon actuation by way of pressing, the inner needle 620 translates to a next position out or exteriorly from the needle portion 600 to dislodge foreign bodies stuck in a lumen of the needle portion 600. Thereafter, the user depresses the injection actuator 410 for delivery and injection of the therapeutic drug into a body (i.e. an eye). The needle portion 620 in the next position exposed away from the needle portion 600 prevents back flow or leakage or incomplete injection being prevented. The injection actuator 410 includes an injection spring 430 in a compressed state upon actuation by way of depressing the injection actuator 410. Upon completion of the delivery of the therapeutic drug, a force from the compressed injection actuator relaxes so that the injection actuator 410 returns back to an original state where the inner needle 620 returns back into the needle portion 600. More intimately, the needle portion 600 retracts away from the body to ensure safe usage and transportation.

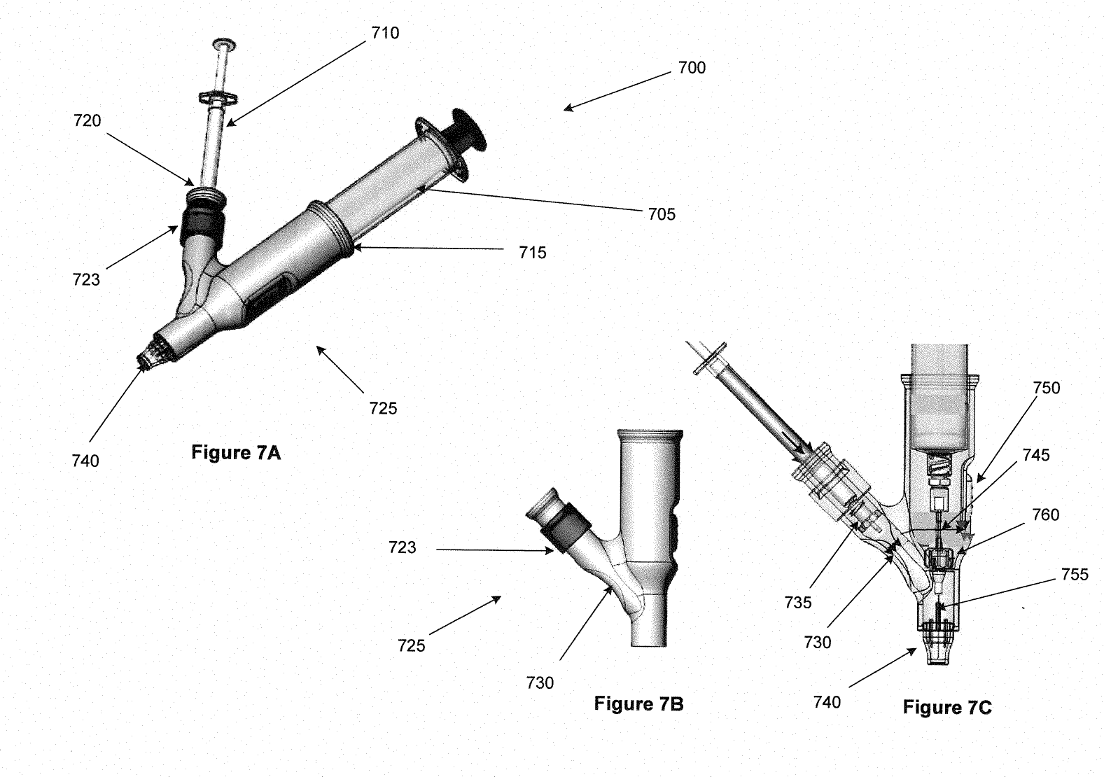

FIGS. 7A to 7C show a further embodiment of the present invention. Here, a housing 725 includes ports for receiving a vacuum source 715 and an injection source 720 for conducting the extraction and injection function. In this embodiment the vacuum source is provided by a syringe 705 mounted to the vacuum port, and the injection source is provided by a second syringe 710 mounted to the injection port. On activation of the vacuum and injection sources, that is pressing the plunger for the injection syringe and withdrawing the plunger of the vacuum syringe, the device effects injection of an agent, or extraction of a sample. The device 725 includes an engagement portion 740 for engaging the eye so as to position the device for optimum extraction of a vitreous sample and injecting an appropriate medicament. Three main purposes/uses of this eye engagement/guard portion:

1. Prevent accidental needle pricks

2. Alignment of 3-4 mm to the limbus of the eye at the pars plana

3. Prevent over-penetration of the needle into the eyeball

4. Prevent the wrong angulation of the needle when it enters the eye

5. Stabilise the eye during injection

Engagement of the injection syringe 710 is through a locking collar 723 which allows a press fit of the syringe 710 into the injection port 720. To release, by sliding the locking collar 723 allowing the removal of syringe 710 from the injection port. Similarly, the vacuum source syringe 705 is pressed fit into the vacuum port 715.

The injection side includes a pinch 730, being a compressible portion of the device acting to mechanically close the channel for selectively sealing communication between the injection source and the needle 755. A pinch release 750, on the vacuum source side of the device, acts as a toggle for the application of the vacuum. When pressed forward, the pinch release 750 closes the vacuum channel 745. The injection pinch can then be released manually allowing the therapeutic to be injected into the sample collection module 760 and thus on injection of the injection syringe 710, the therapeutic is injected through needle 755 into the eye. When a vitreous sample is to be extracted, the pinch release 750 is released, and the injection pinch 730 squeezed and thus allowing a sample to be drawn through the needle 755 subsequently through the sample collection module 760.

Thus, the device 725 provides for the core invention, in that it allows a single system to both extract a sample and inject a medicament within the same device.

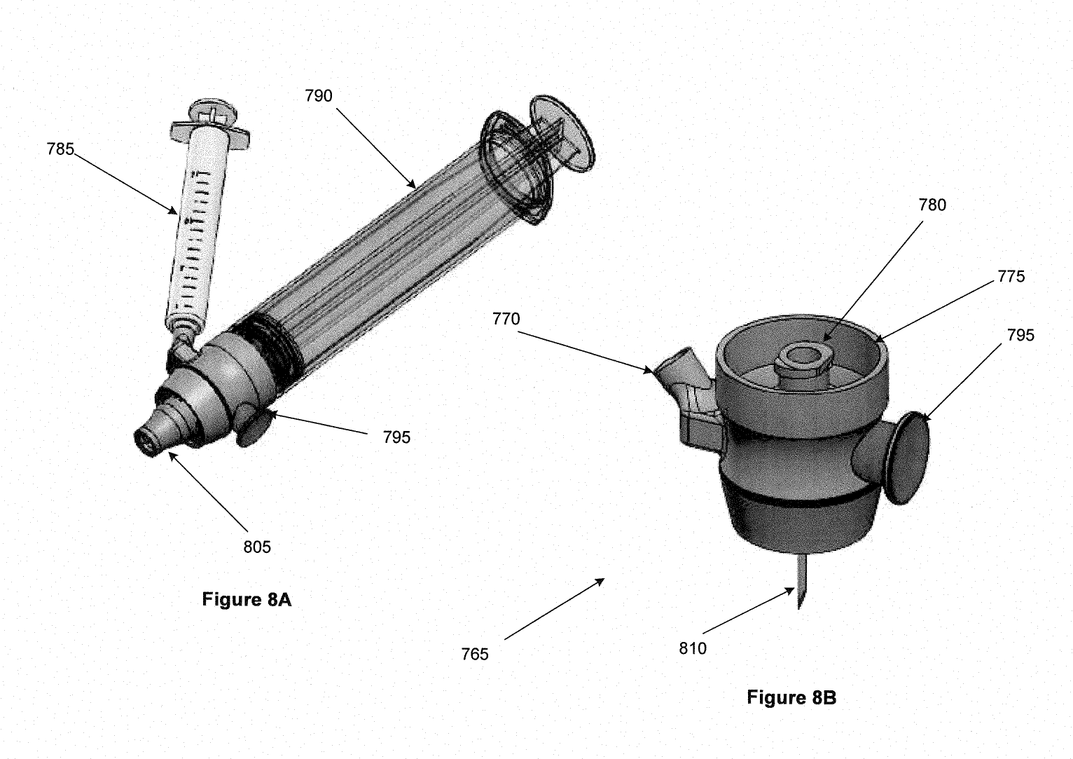

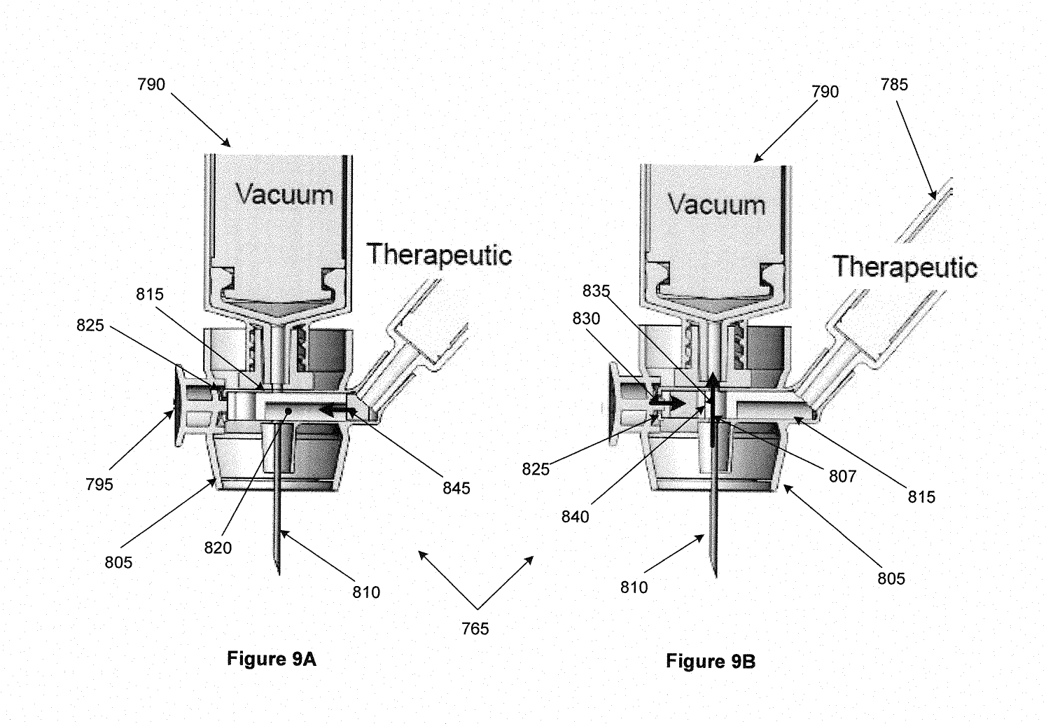

FIGS. 8A, 8B, 9A and 9B show an alternative embodiment of the present invention. Here, an injection and extraction ophthalmic device comprises a housing 765 having a port 775 for receiving a vacuum source, in particular, a syringe whereby the tip enters a further recess 780. There is a second port 770 for receiving a therapeutic input which in this case is a second syringe 785 containing the therapeutic agent. The housing 765 further includes a retractable portion 805 for engaging the eye and a needle 810 which projects from, or is held within, the housing depending upon the position of the retractable portion 805. The housing 765 further includes a button 795 for selectively placing either the therapeutic syringe 785 or the vacuum syringe 790 in fluid communication with the needle 810.

With particular reference to FIGS. 9A and 9B, the housing 765 includes a sample collection module 815 which is movable between two positions by depressing the button 795 against a spring 825. When the spring 825 is in a relaxed position the sample collection module 815 is in a first position and provides a channel 820 providing the fluid communication between the therapeutic syringe 885 and needle 810. Therefore the operator can inject 845 the therapeutic agent directly into the eye in the position as shown in FIG. 9A.

FIG. 9B show the condition when the button 795 is depressed and therefore moving the channel 820 into a second position. In this position a bore 840 places the vacuum syringe 790 in fluid communication with the needle 810 and thus allowing a sample to be drawn 835 into the sample collection module 815 for subsequent testing. Given the gel-like viscosity of the vitreous humor, as the sample is drawn in, it is separated from the rest of the vitreous, to reduce the risk of traction on the retina. This reduced risk of traction, may potentially reduce the risk of complications such as retinal tears and retinal detachments when the needle is removed. This extraction and separation of the sample is demonstrated as the sample collection module 815 acts as a shearing device as it is biased back to the first position the excess vitreous humor, shearing at the interface 807 between the sample collection module 815 and the rest of the housing 765, separating the extracted sample from the rest of the vitreous.

Thus, the device has the potential to both inject a therapeutic agent and extract a vitreous sample using the same device. The embodiment of FIGS. 8A, 8B, 9A and 9B allows this to be achieved using a single needle as compared to the earlier embodiments.

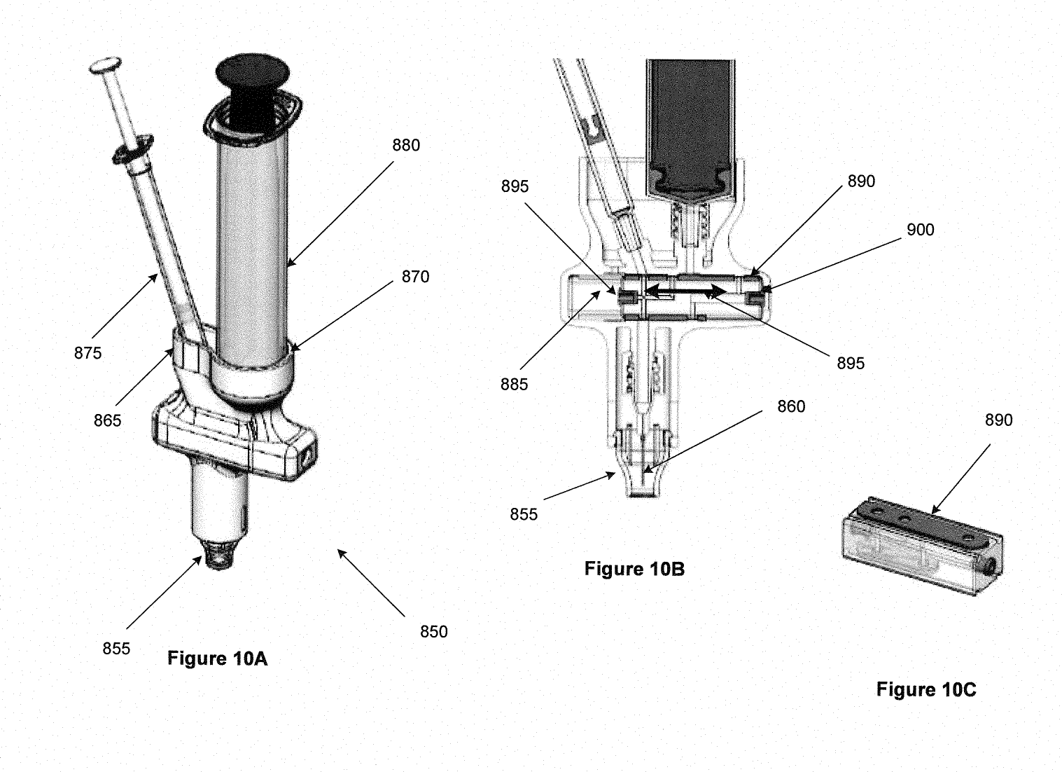

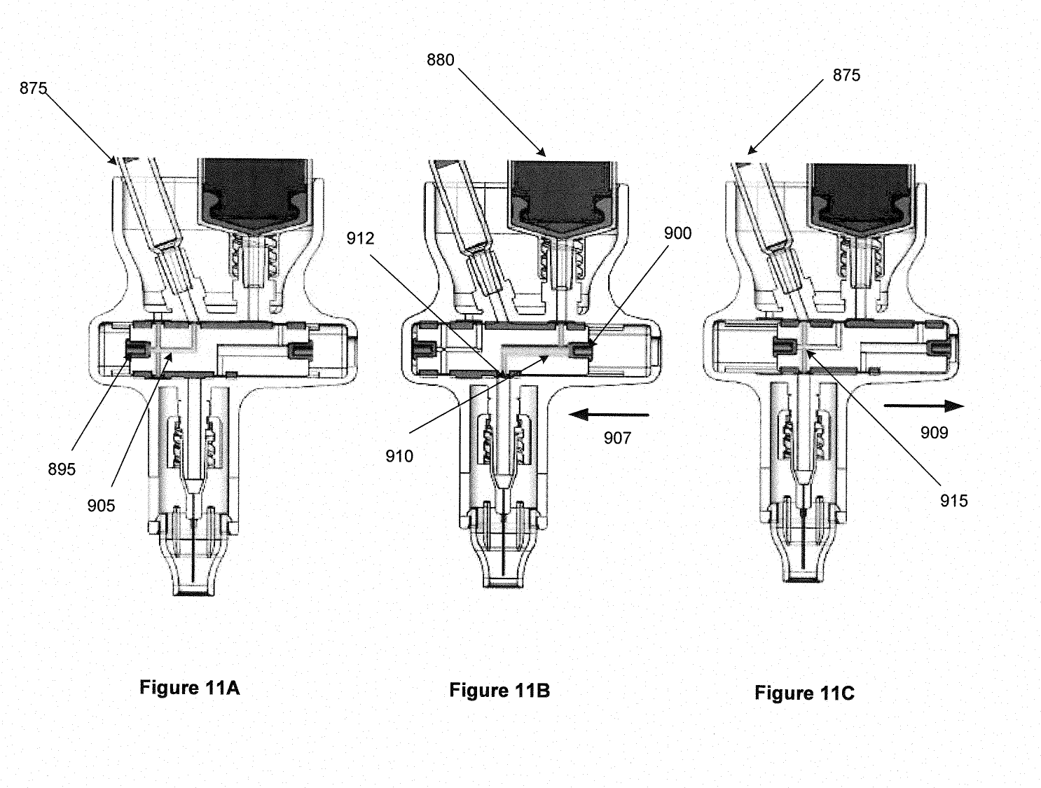

FIGS. 10A to 10C and 11A to 11C show a further embodiment of the present invention. Here a device includes a housing 850 arranged to engage a vacuum source, in this case a syringe 880, and a therapeutic injection source, in this case a syringe 875. These are respectively engaged in ports 870 and 865 so as to be in selective fluid communication with a needle 860. The housing 850 includes a retractable location device 855 for locating the housing 850 on the eye which can then be further retracted so as to have the needle 860 project from the housing and penetrate the eye.

A void 885 within the housing 850 allows for a sample collection module 890 to move between three positions within the void 885 to selectively place either the vacuum source 880 or injection source 875 in fluid communication with the needle 860. The sample collection module 890 includes ports 895, 900 for inserting 895 and extracting 900 components within the slide for their selective use.

FIGS. 11A to 11C show the housing in cross section with the difference between FIGS. 11A, 11B and 11C depending upon the position of the sample collection module within the space of the housing. The position shown in FIG. 11A, places the sample collection module such that it seals the vacuum source 880, but allows the injection channels 905 to be in communication with the injection source 875. In this position, the sample collection module is primed by the injection syringe with the injectable therapeutic prior to injection into the eye, preventing the injection of air. It will be appreciated that the device may use a dedicated priming source in order to prime the sample collection module. For consistency, the position shown in FIG. 11A will be nominated as the third, or priming, position.

FIG. 11C shows the position of the sample collection module having moved 909 to the right, whereby the injection channel 915 is in fluid communication with both the injection source 875 and the needle 860 so as to inject the primed therapeutic into the eye. Once again, for consistency, this is nominated as the first, or injecting, position.

In the position shown in FIG. 11B, the sample collection module is moved 907 to the left of the priming position, placing sample channels 910 and the vacuum source 880 in fluid communication with the needle. This allows for a sample of the vitreous to be extracted as a result of the vacuum source and drawn into the sample channel 910. This position is nominated as the second, or extracting, position.

As mentioned, the gel-like consistency of the vitreous humor tends to draw excess material from the eye. To prevent this leading to further damage, the sample collection module is moved back to the priming position. This has the effect of shearing the excess vitreous humor at the interface 912 between the sample collection module and the rest of the housing. Thus the sample collection module acts as a shearing device for separating the sample to be collected from the excess vitreous humor.

On completion of the injecting and sampling steps, the sample collection module 890 can then be removed and sent for diagnostic tests of the sample.

1. An extraction and injection ophthalmic device comprising;
a housing having;
an injection port arranged to receive an injection source;
a vacuum port arranged to receive an vacuum source;
an eye engagement portion, and;
at least one needle for selective fluid communication with one or both of the vacuum port or injection port;
wherein on activation of the vacuum source the device is arranged to extract a sample through the needle, and on activation of the injection source the device is arranged to inject an agent through the needle.

2. The device according to claim 1, further including a sample collection module engaged with the housing, the sample collection module arranged to receive the extracted sample.

3. The device according to claim 1, further including a shearing device arranged to shear excess vitreous humor from the sample to be extracted.

4. The device according to claim 2, wherein the sample collection module is selectively removable from the housing.

5. The device according to claim 1, wherein the sample collection module is engaged fully within the housing.

6. The device according to claim 1, wherein the injection source includes a syringe.

7. The device according to claim 1, wherein the vacuum source includes any one of: a syringe, a vacutainer or a pump.

8. The device according to claim 1, wherein the device includes one needle in selective fluid communication with the injection source or the vacuum source.

9. The device according to claim 8, further including a toggle to switch between a first and second position corresponding to fluid communication between the injection source and the vacuum source, respectively.

10. The device according to claim 9, wherein the toggle is further arranged to switch to a third position corresponding to fluid communication with a priming source.

11. The device according to claim 10, wherein the priming source includes the injection source.

12. The device according to claim 9, wherein the toggle is arranged to move the sample collection module when switching from fluid communication between the injection source and the vacuum source.

13. The device according to claim 9, wherein the sample collection module is arranged to act as the shearing device as it moves from the second position to the first or third position.

14. The device according to claim 1, wherein the needle is fixed relative to the housing, with the eye engagement portion movable between an extended position covering the at least one needle and a retracted position exposing the needle.
